(19) United States
(12) Patent Application Publication
(10) Pub. No.: US 2026/0096774 A1
(43) Pub. Date: Apr. 9, 2026

(54) DENTAL IMPLANT STABILITY ANALYZERS

(71) Applicants: University of Washington, Seattle, WA (US); Quiver Dental, Inc., Seattle, WA (US)

(72) Inventors: I-Yeu Steve Shen, Seattle, WA (US); Yifeng Liu, Torrance, CA (US); Johnny Wang, Seattle, WA (US); Weiwei Xu, Seattle, WA (US); Michel Dard, Seattle, WA (US)

(21) Appl. No.: 19/353,595

(22) Filed: Oct. 8, 2025

Related U.S. Application Data
(60) Provisional application No. 63/705,273, filed on Oct. 9, 2024.

(51) Int. Cl. A61B 5/00; A61B 5/11; A61C 8/00; A61C 19/04

(52) U.S. Cl. CPC A61B 5/4851; A61B 5/002; A61B 5/1111; A61B 5/682; A61B 5/6838; A61C 8/0093; A61C 19/04; A61B 2560/0214; A61B 2562/0219

(57) ABSTRACT

An intraoral sensor comprising a clamp and a motor-sensor unit. The clamp comprising an upper portion and a lower portion. The lower portion comprising a plurality of holes for receiving forceps tips, a first opening and a second opening, and an engagement portion for contacting an implant. The motor-sensor unit comprising a force generator, a sensor, and a clip connecting the force generator and the sensor. The intraoral sensor is configured to be retained on the implant to generate a measurement of implant stability. The first portion of the motor-sensor unit is received in the first opening of the lower portion of the clamp. The second portion of the motor-sensor unit is received in the second opening of the lower portion of the clamp. The motor-sensor unit is removably connected to the clamp.

### INCORPORATION BY REFERENCE TO ANY PRIORITY APPLICATIONS

This application claims the priority benefit of U.S. Provisional Application No. 63/705,273 filed Oct. 9, 2024, the entire contents of which is hereby incorporated by reference herein.

### BACKGROUND

The present disclosure generally relates to an intelligent device and systems for use with dental implants, and more particularly, for measuring the stability of a dental implant over its life cycle. The stability of a dental implant is used to determine the immobility of an implant in bone which can be an indication of osseointegration.

### SUMMARY

In some aspects, an intraoral sensor is disclosed. The intraoral sensor can include a clamp and a motor sensor unit. The clamp can include an upper portion and a lower portion. The lower portion can include a plurality of holes for receiving forceps tips, a first opening and a second opening, and an engagement portion for contacting an implant. The motor sensor unit can include a force generator, a sensor, and a clip connecting the force generator and the sensor. The intraoral sensor can be configured to be retained on the implant to generate a measurement of implant stability. The intraoral sensor can include a first portion of the motor-sensor unit that is received in the first opening of the lower portion of the clamp and a second portion of the motor-sensor unit that is received in the second opening of the lower portion of the clamp. The motor-sensor unit can be removably connected to the clamp.

The intraoral sensor can include one or more of the following features. The motor-sensor unit can be secured to the clamp by an interference fit. The intraoral sensor can include a pair of claws for engaging the implant. The motor-sensor unit can comprise at least one protrusion forming an axial stop, wherein the axial stop and the pair of claws are configured to secure the implant. The force generator can be a motor. The sensor can be an accelerometer. The implant can be an abutment or a crown. The force generator can be oriented to pass through a center of the pair of claws. The motor-sensor unit can include a clip extending between the force generator and the sensor, wherein the motor-sensor unit is elastic and allows the motor-sensor unit to open with the clamp. The clip of the motor-sensor unit can be preloaded to help retain the motor-sensor unit on the clamp. The upper portion of the clamp can be sufficiently tall to receive a pair of forceps while lowering a force required to open the pair of forceps. The claws of the clamp can have an engagement portion that have a pair of straight edges joined at an obtuse angle, wherein the engagement portion is configured to accommodate an implant with a wide range of diameters. The intraoral sensor can further include an electrical cable extending from the sensor.

In some aspects, a system for measuring implant stability is disclosed. The system can include an intraoral sensor, a controller, and an application for receiving an implant stability measurement form the intraoral sensor. The intraoral sensor can include a clamp and a motor-sensor unit. The clamp can include a pair of claws for engaging an implant. The motor-sensor unit can include a force generator and a sensor. The motor-sensor unit can be removably connected to the clamp. The controller can be configured to drive the force generator and the sensor.

The system for measuring implant stability can include one or more of the following features. The system can further comprise a wired connection between the intraoral sensor and the controller, and wherein the controller further supplies power to the intraoral sensor. The intraoral sensor can comprise a power source and a Bluetooth chipset; wherein the Bluetooth chipset is configured to transmit wirelessly with the controller.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

FIG. 1 illustrates a cross-sectional view of an implant with a healing abutment attached over the implant.

FIG. 2 illustrates an exploded view of an implant with a crown attached to the implant.

FIG. 3 illustrates an embodiment of an architecture of an implant stability analyzer system.

FIG. 4 and FIG. 5 illustrate embodiments of a dental implant stability analyzer.

FIGS. 6A and 6B illustrate enlarged views of the engagement portions of the claws of a lower portion of the clamp of the embodiment of the dental implant stability analyzer of FIGS. 4 and 5.

FIG. 7 illustrates an embodiment of the dental implant stability analyzer of FIGS. 4 and 5 before it engages an abutment or crown.

FIG. 8 illustrates an embodiment of the dental implant stability analyzer of FIGS. 4 and 5 after it engages an abutment or crown.

FIG. 9 illustrates another embodiment of a dental implant stability analyzer.

FIG. 10 illustrates an enlarged view of the motor sensor unit of the dental implant stability analyzer of FIG. 9.

FIG. 11 illustrates another embodiment of a dental implant stability analyzer.

FIG. 12 illustrates a top view of the dental implant stability analyzer of FIG. 11.

FIGS. 13A-13D illustrate a plurality of views of a clamp of another embodiment of a dental implant stability analyzer.

FIGS. 14A-14C illustrate a plurality of views of the dental implant stability analyzer with the clamp of FIGS. 13A-13D.

FIG. 15A illustrates another embodiment of a dental implant stability analyzer.

FIG. 15B illustrates another embodiment of a clamp that can be used in the dental implant stability analyzer of FIG. 15A.

FIG. 15C illustrates an embodiment of a cross-sectional view of the dental implant stability analyzer of FIG. 15A.

FIG. 15D illustrates of a cross-sectional view of the housing of the motor-sensor unit of the dental implant stability analyzer of FIG. 15A.

FIG. 15E illustrates the electronics of the motor-sensor unit of the dental implant stability analyzer of FIG. 15A.

FIG. 16 illustrates an exploded view of the components of the dental implant stability analyzer of FIG. 15A.

FIG. 17 illustrates an embodiment of a standard specimen used to evaluate the accuracy of a clamp and motor-sensor unit.

FIG. 18 illustrates an embodiment of a standard procedure to calibrate a clamp and motor-sensor unit using resonance frequency.

FIG. 19 illustrates an embodiment of a flowchart of a self-diagnostic test.

FIG. 20 illustrates a sample accelerometer measurement.

FIG. 21 illustrates another embodiment of a flow chart of a self-diagnostic test.

### DETAILED DESCRIPTION

The present disclosure may be understood more readily by reference to the following detailed description of embodiments of the disclosure and the examples of devices configured to measure the stability of medical implants. The following description, along with the accompanying drawings, sets forth certain specific details to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to communication systems and networks, have not been shown or described to avoid unnecessarily obscuring descriptions of the embodiments.

The present disclosure is directed to surgical procedures, for example, dental procedures like placement of a dental implant which term includes reference to the surgery and associated implantable medical devices such as a dental implant (e.g., implant, crown, and abutment). In some embodiments, the presently disclosed device can be directed to measuring the stability of other medical implants (e.g., spinal screws, etc.).

Embodiments disclosed herein relate to dental implant stability analyzers, systems including the same, and methods of making and using the same. The dental implant stability analyzers disclosed herein can allow dental implant stability analyzers to be directly attached onto the dental implant (e.g., implant, crown, and/or abutment). Measuring the stability of an abutment facilitates measurements of pre-crown stability, whereas measuring the stability of the crown facilitates measurements of post-crown stability. The disclosed implant stability analyzer can provide faster, easier, and safer stability measurements as portions of the implant (e.g., crown or abutment) need not be removed.

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

### Overview

Dental implant stability refers to the immobility of a dental implant in surrounding bone. High implant stability is an indicator of successful osseointegration, which can manifest in strong functional and structural connection between the implant and the bone. Implant stability (e.g., dental implant stability) provides a reflection of the well-being of an implant (e.g., dental implant) throughout its life cycle.

In implant dentistry, the standard of care is to first place an implant in bone. After the implantation, a medical professional will evaluate the implant stability. If the stability is determined to be low, the medical professional can use a healing cap to seal the top of the implant and suture the gingiva. This ensures that the implant receives no external disturbance as the bone heals. However, if stability is determined to be high, a healing abutment can instead be attached over the implant to shape the gum tissue. FIG. 1 illustrates a cross-sectional view of an enlarged view 100 of teeth anatomy during implant treatment with a healing abutment 104 attached over the inserted implant 102. As discussed above, when implant stability is low, a medical professional can instead use a healing cap (not shown) that will be replaced by the healing abutment 104 when the implant improves in stability.

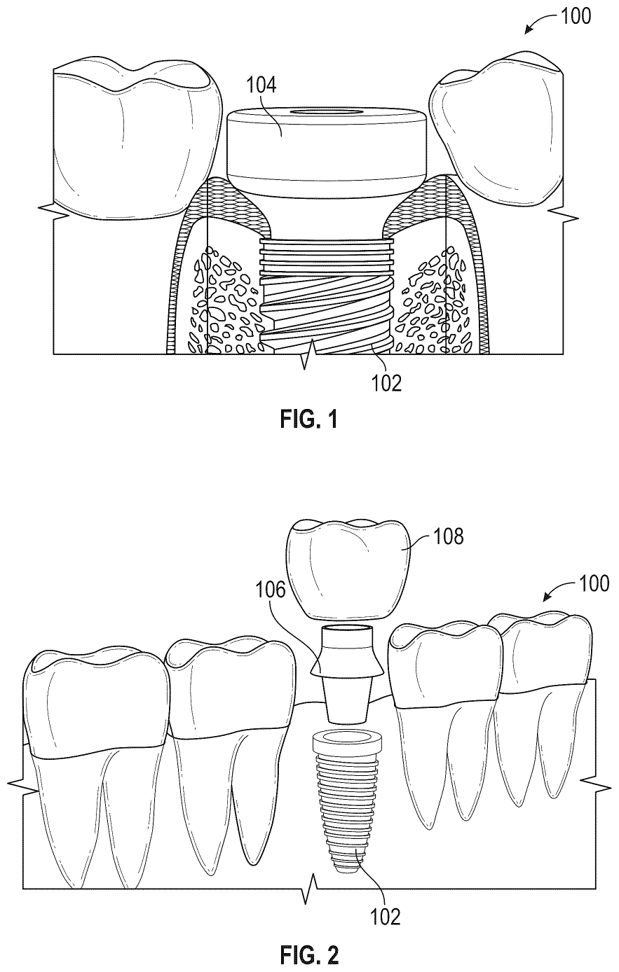

FIG. 2 illustrates a view of the gum line 100 once the bone around the implant has healed. Once the bone heals, a medical professional can replace the healing abutment 104 with a regular abutment 106. A crown 108 can be attached to the abutment 106 in order to receive bite loads and allow the user to regain use of the tooth. In some embodiments, before placing the crown 108, a medical professional must assure in a pre-restoration checkup that the implant 102 has sufficient stability. Failure to do so can result in complications such as pain, bone loss, or even implant failure. This can frequently occur after crown placement. If a medical professional finds that there is low implant stability before crown placement, the medical professional may choose to let the bone heal longer or take remedial actions, including removal of the implant. For the purposes of this application, implant stability measured before crown placement will be referred to as “pre-crown stability.”

After a successful crown placement, implant stability can be measured regularly through the implant's life cycle. Known as implant maintenance, this practice allows a medical professional to monitor the bonding strength between the implant and the surrounding bone. Regular implant maintenance can provide an effective method of identifying peri-implantitis and the lose of bone-impact contact. For the purposes of this application, implant stability measured after crown placement will be referred to as “post-crown stability.”

At this time, many medical professional rely on subjective measure to evaluate pre-crown implant stability. These can include, for example, visual inspection, manual inspection (e.g., touch of hands), X-ray imaging, or torquing the implant. However, these existing methods can be inaccurate (e.g., false negatives) or reliant on subjective standards. These subjective standards are based on a judgment call where success is largely dependent on the medical professional's experience. As no standard of care exists, medical professional lack effective tools to properly evaluate post-crown stability. This can result in unexpected implant failure with no early visual signs of deterioration.

Resonance frequency analysis (RFA) is a method to monitor implant stability. However, RFA can only measure pre-crown stability. In a RFA, the crown or abutment is first removed to expose the implant. A sensor unit of the RFA device is then screwed onto the implant for a firm attachment. A probing section of an instrument is then brough in close proximity to the sensor unit to measure the stability. Use of RFA to monitor implant stability can pose many challenges—in particular the direct connection of the sensor unit onto the implant. First, the removal (and subsequent restoration) of the abutment can be a cumbersome process. If the RFA indicates low stability, the abutment must be restored for the bone to heal better. Second, the removal of the abutment involves a risk of accidentally disturbing the implant, raising a safety concern. Third, because implants under different manufacturers may have different threads and dimensions, a sensor unit cannot be used interchangeably among different brands of implants. The lack of interchangeability requires that manufacturers and users maintain a wide selection of sensor units to accommodate different implant brands, resulting in large design, manufacturing, and inventory costs. Lastly, RFA cannot be used to measure post-crown stability as a direct connection of a sensor unit to the implant is not viable for post-crown stability measurements. Once a crown is permanently attached to the implant, the crown and its supporting abutment cannot be removed.

Another method of measuring implant stability is to directly attach a sensor unit to the abutment or crown. In this method, because no crown or abutment removal is needed, this can be an easier and safer method than RFA for performing stability measurements.

However, attaching a sensor unit onto an abutment or a crown, can pose its own challenges. First, abutments and crowns have various shapes and sizes. For example, the abutment can take a conical or goblet shape. The abutment diameter can range from 3 to 7 mm depending the location of the implant. The abutment can have a wide range of heights to accommodate a wide range of patient conditions. This can therefore produce a wide range of aspect ratios. As a result, the sensor unit must have an effective mechanism to attach and remove the sensor unit to a wide variation of abutment shapes and sizes and to remove the sensor unit therefrom.

Second, attaching the sensor unit directly to the abutment or crown can be problematic given the limited space in a patient's mouth. In the mesial-distal direction, a tooth width is between about 10-12 mm for molars, between about 8-9 mm for premolars, and between about 5-6 mm for incisors. In the buccal-lingual direction, available space is only about 10-15 mm before touching the tongue or the cheek. Normal tooth height is normally between about 7-8 mm and 10 mm when the gum line has receded. Above the molars, there is only limited space of around 20 mm before reaching the mating molars at a mandible or maxilla. There is therefore very limited inter-dental space that the sensor unit can fit in.

Third, there is an asymmetric force requirement for directly attaching a sensor unit to the abutment or crown. It is desirable to use a relatively small grip force to open the sensor unit for attachment onto the abutment. However, once it is attached, the sensor unit needs a sufficiently large enough clamping force to keep the sensor unit attached to the abutment.

Another challenge is a sufficiently clear line of sight when deploying the sensor unit onto the target abutment. This is especially important for molars, because they are difficult to see. When a missing molar is replaced by an implant with an abutment, the teeth adjacent the missing molar can be much taller than the abutment. Therefore, users often rely on a clear line of sight to locate the abutment to deploy the sensor unit thereon. It is desirable that the sensor unit itself not block the line of sight of the abutment.

Lastly, another challenge of attaching the sensor unit directly to the abutment or crown is the case of providing an effective infection control strategy. On the one hand, if the sensor unit is disposable, the system for measuring implant stability can be very expensive to operate. However, if the sensor unit is reusable, the sensitive sensor elements may not be able to survive steam sterilization in autoclaves. The electronics of the sensor unit is generally sensitive, while the external structure must be sturdy and strong in order to firmly attach to the abutment.

The presently disclosed system, device, and method provide for a method of measuring pre-crown and post-crown implant stabilities that improve upon existing methods while addressing the aforementioned challenges.

### Overview of Dental Implant Stability Analyzer

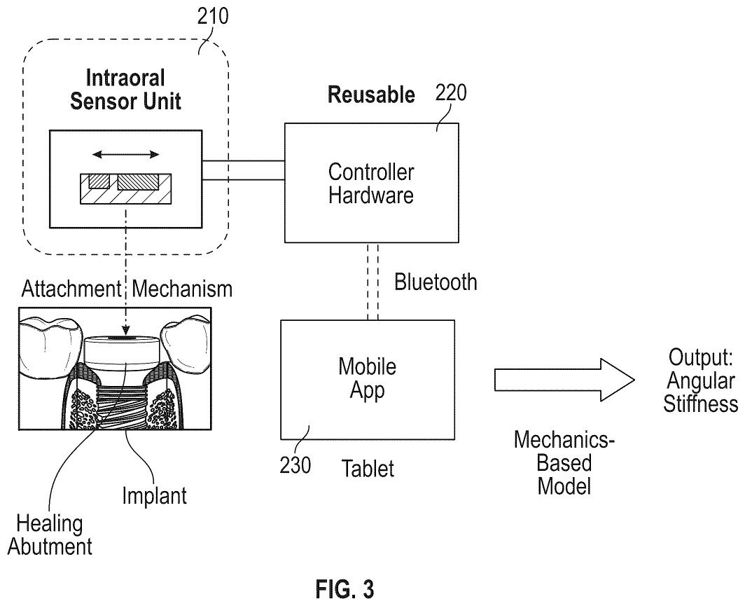

FIG. 3 illustrates an embodiment of a architecture 200 of an implant stability analyzer system for measuring pre-crown and/or post-crown implant stability. In some embodiments, the architecture 200 can include an intraoral sensor unit 210, a controller unit 220, and an application 230 for receiving implant data. In some embodiments, the intraoral sensor unit 210 can be a one-time use consumable intraoral sensor unit 210. In some examples, the controller unit 220 can be reusable. In some embodiments the application 230 can be a mobile application that is used on an electronic device (e.g., cell phone, tablet, etc.). In some embodiments, the intraoral sensor unit 210 comprises an attachment mechanism, a motor-sensor unit (MSU), and an electrical cable.

Embodiments of the intraoral sensor unit will be described in more detail below. In some embodiments, sensor unit can include an attachment mechanism. In some examples, the attachment mechanism can be a quick-connect-fast-release mechanism such as a clamp for attaching to the abutment and/or crown of the implant. In some embodiments, the sensor unit includes a MSU that comprises at least one of a force generator (e.g., a motor) and a sensor. In some examples, the MSU comprises a structure for attaching the MSU to the attachment mechanism of the sensor unit. In some embodiments, the sensor unit can include a power source configured to provide power to the at least one force generator and the sensor. In some examples, the power source can comprise a battery, a capacitor, an input that can be connected to a battery, an electrical outlet that is spaced from the sensor unit, or combinations thereof. In some embodiments, the power source is attached to the sensor unit directly or indirectly (e.g., electrical cable or induction link respectively).

### Method of Measuring Stability Using the Dental Implant Stability Analyzer

In some embodiments, implant stability is measured by gently moving the implant and sensing its motion through the healing abutment or crown. With the presently disclosed dental implant stability analyzer, the sensor unit is first attached to the healing abutment or crown. The sensor unit can then by driven by the controller hardware to wiggle the implant through the healing abutment or crown and detect the motion generated. The measured motion (e.g., acceleration) can then be converted into an indicator of stability (e.g., angular stiffness) through a mechanics-based model in the application (e.g., a mobile application). In some embodiments, the mechanics-based model is experimentally validated in benchtop tests before it is used in the mobile application.

### Embodiments of Intraoral Sensor Unit

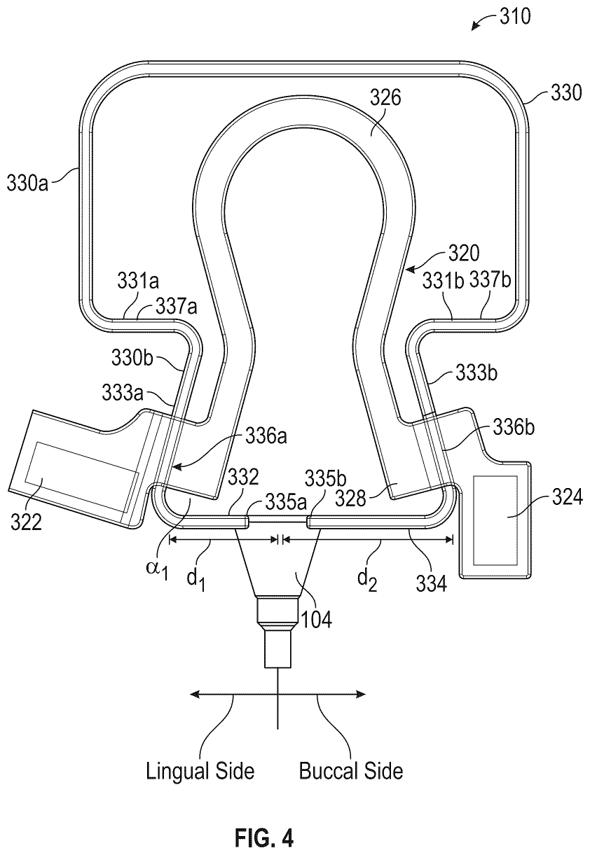

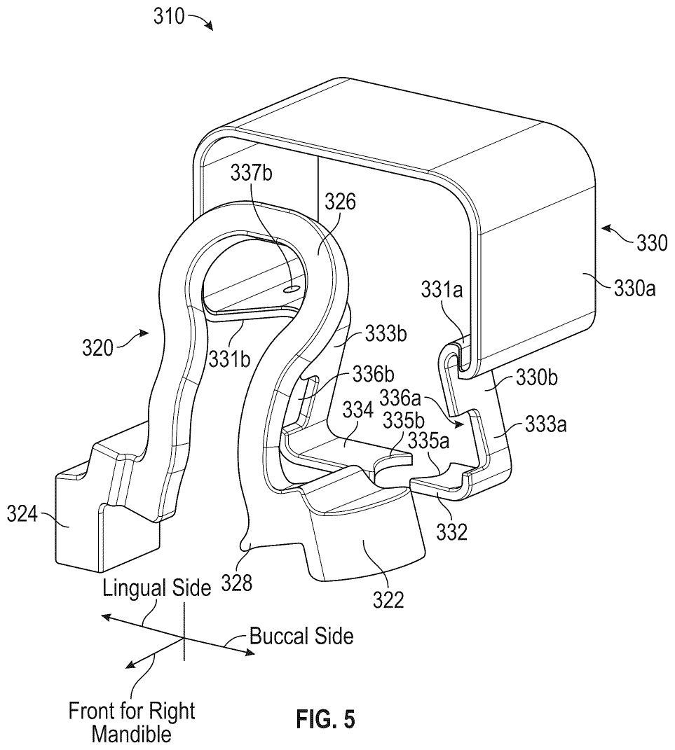

FIG. 4 illustrates an embodiments of a sensor unit 310. In some embodiments, the sensor unit 310 comprises a clamp 330, a MSU 320, and an electrical cable (not shown). FIG. 5 illustrates an exploded view of the sensor unit 310 to illustrate how the clamp 330 and the MSU 320 are configured to engage.

In some embodiments, the clamp 330 of the sensor unit 310 has an inverted-U shape. In some examples, the clamp 330 includes an upper portion 330a and a lower portion 330b.

In some embodiments, the upper portion 330a has the shape of an open rectangular box. In some embodiments, the upper portion 330a has a height of about 5 mm, about 10 mm, about 15 mm, about 20 mm, about 25 mm, between about 5 mm and about 25 mm, between about 10 mm and about 20 mm, and any value in between those ranges listed, including endpoints. In some examples, the height of the upper portion 330a ensures that the upper portion 330a is unlikely to touch a mating tooth in the mandible or maxilla if the sensor unit 310 is deployed to a molar position. In some embodiments, the height of the upper portion 330a is sufficiently large to create two rectangular regions between the clamp 330 and the MSU 320. As will be discussed in more detail below, the two rectangular regions can allow a pair of forceps to access and engage a pair of holes 337a, 337b on a portion of the lower portion 330b to open or release the clamp 330. In some examples, the vertical walls of the upper portion 330a serve as lever arms when the clamp 330 is opened. The height of the upper portion 330a can therefore determine the force Fₒₚₑₙ to open the clamp 330. In some embodiments, the ratio of the height of the upper portion 330a to the lower portion 330b can determine the clamping force F_{claw} at the clamp 330 after the clamp 330 is attached onto an abutment or crown, wherein the total height of the clamp 330 forms the length of the lever arm. In some embodiments, the height ratio can be adjusted to provide a reasonable opening force Fₒₚₑₙ can be achieved by a hand grip (e.g., 40 Newtons) to produce a relatively large clamping force (e.g., 24 Newtons) at the clamp 330. In some examples, the height of the upper portion 330a provides the clamp 330 with sufficient height to receive a pair of forceps. In some embodiments, the height of the upper portion 330a is designed to lower the force needed to open the forceps. In some embodiments, the widened side walls of the upper portion 330a are positioned above neighboring teeth and are configured to allow the clamp 330 to generate a sufficiently large force to clamp onto an abutment or crown.

The lower portion 330b can form the engagement portion of the clamp 330. In some embodiments, the lower portion 330b comprises an upper first portion 331a, 331b, a middle second portion 333a, 333b, and a lower portion that forms a claw 332, 334. In some embodiments the lower portion 330b is formed from two claws—the claw 332 and the claw 334. The claw 332 and the claw 334 can be designed to attach the clamp 330 to an abutment or crown. In some embodiments, the lower portion 330b has a height of about 5 mm, about 10 mm, about 15 mm, between about 5 mm and about 15 mm, between about 6 mm and about 14 mm, between about 7 mm and about 13 mm, between about 8 mm and about 12 mm, between about 9 mm and about 11 mm, and any value in between those ranges listed, including endpoints.

In some embodiments, the width of the lower portion 330b in the mesial-distal direction is narrower than the upper portion 330a. In some embodiments the width of the lower portion 330b is about 1 mm, about 2 mm, about 3 mm, about 4 mm, about 5 mm, about 6 mm, about 7 mm, about 8 mm, about 9 mm, about 10 mm, between about 1 mm and about 9 mm, between about 2 mm and about 8 mm, between about 3 mm and about 7 mm, between about 4 mm and about 6 mm, and any value in between those ranges listed, including endpoints. In some examples, the narrower width of the lower portion 330b can allow the lower portion 330b to fit in the width of a missing tooth, regardless of whether the tooth is a molar, a premolar, or an incisor.

In some embodiments, the first portion 331a and the first portion 331b form a portion of the lower portion 330b that is attached to a portion of the upper portion 330a. In some examples, each of the first portion 331a and the first portion 331b can include a hole 337a and hole 337b respectively on the flat surface of the first portion 331a and first portion 331b. In some examples, the holes are provided to receive the tips of a pair of forceps to open and release the clamp 330. In some embodiments, the first portion 331a and the first portion 331b are trapezoidal in shape and extend the medial-distal width of the upper portion 330a to about 10 mm. In some embodiments, the position of the hole 337a and hole 337b can ensure that, when the forceps are used to open the clamp 330, only the upper portion 330a is stressed while keeping the lower portion 330b stress-free. In this way, the lower portion 330b will not be stressed until the claw 332 and the claw 334 are engaged with the abutment. In some examples, the location of the hole 337a and the hole 337b are designed to allow a small force to open the forceps.

In some embodiments, the middle second portion 333a and second portion 333b form an angled portion of the lower portion 330b that connects the first portion 331a and first portion 331b with the claw 332 and claw 334 respectively. In some embodiments, the first portion 331a and the first portion 331b can form an angle α₁ with the claw 332 and claw 334 respectively. In some embodiments the angle α₁ can be about 45°, about 50°, about 55°, about 60°, about 65°, about 70°, about 75°, about 80°, about 85°, about 90°, between about 45° and about 90°, between about 50° and about 85°, between about 55° and about 80°, and any value in between those ranges listed, including endpoints. In some embodiments, the purpose of the angle is to allow the formation of a pair of flat surfaces (e.g., the claw 332 and claw 334) that serves as transitions to the upper portion 330a. In some embodiments, the second portion 333a and the second portion 333b include an opening 336a and an opening 336b respectively. The opening 336a and the opening 336b are configured to receive a portion of the MSU 320 and can precisely define the position of the MSU 320 on the sensor unit 310. In some embodiments, the opening 336a and the opening 336b can have a length of about 1 mm, about 1.2 mm, about 1.4 mm, about 1.6 mm, about 1.8 mm, about 2 mm, about 2.2 mm, about 2.4 mm, about 2.6 mm, about 2.8 mm, about 3 mm, about 3.2 mm, about 3.4 mm, about 3.6 mm, about 3.8 mm, about 4 mm, about 4.2 mm, about 4.4 mm, about 4.6 mm, about 4.8 mm, about 5 mm, between about 1 mm and about 5 mm, between about 2 mm and about 4 mm, any value in between those ranges listed, including endpoints.

In some embodiments, the lower portion 330b is asymmetric. For example, the claw 332 on the lingual side is shorter than the claw 334 on the buccal side. This asymmetry is designed to accommodate a motor 322 in the MSU 320 that may be larger than a sensor 324. In some embodiments, the unequal lengths of the claw 332 and the claw 334 can allow the clamp 330 and MSU 320 assembly of the sensor unit 310 to have equal lengths in the buccal and lingual sides. In some examples, the asymmetry of the claw 332 and the claw 334 can enable a user to quickly identify the lingual side and the buccal side as the lengths are unequal. This can help to reduce user error when assembly the MSU 320 and the clamp 330, but also when deploying the sensor unit 310 onto the abutment or crown.

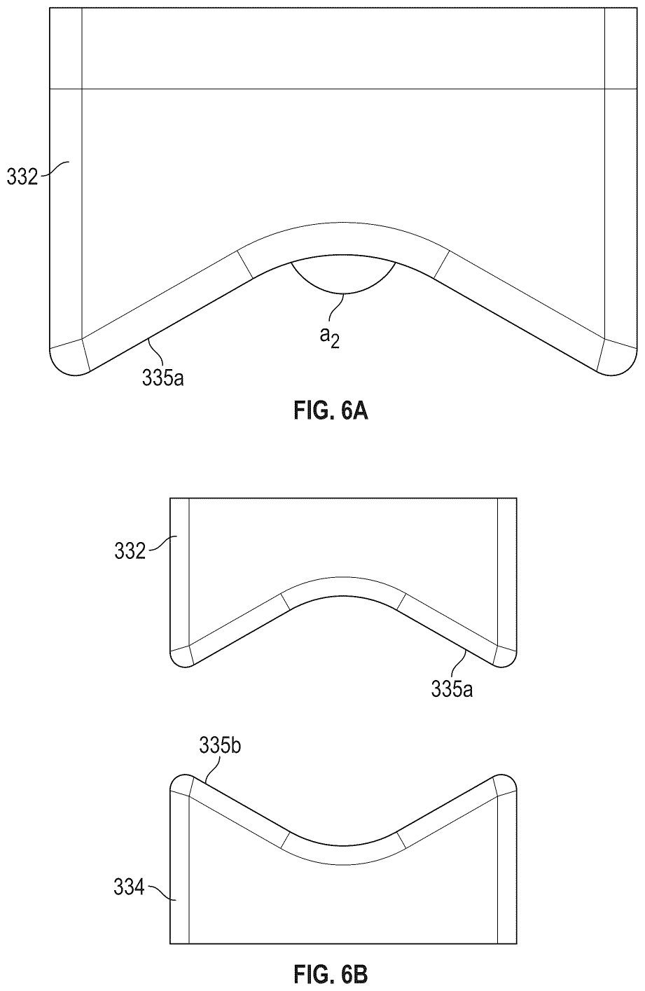

FIGS. 6A and 6B illustrate enlarged views of engagement portion 335a and engagement portion 335b of the claw 332 and claw 334 respectively. As shown in FIG. 6B, each of the engagement portion 335a and the engagement portion 335b can include two straight edges. In some embodiments, each of the two straight edges of the engagement portion 335a and engagement portion 335b are joined by a smooth corner in order to reduce any possible stress concentration. In some embodiments, the two straight edges form an obtuse angle α₂ that is greater than 90°, about 120°, about 150°, less than 180°, between 90° and 180°, between about 100° and about 150°, and any value in between those ranges listed, including endpoints. In some embodiments, an obtuse angle is beneficial as it can allow the opening of the clamp 330 less sensitive to the diameter of the abutment.

The materials and dimensions of the clamp 330 can determine how wide the clamp 330 can open without yielding the clamp material. In some examples, the length of the clamp 330 is large enough to allow a forceps to access the hole 337a and hole 337b of the lower portion 330b. In some embodiments, the length of the clamp 330 in the buccal-lingual direction is about 15 mm, about 20 mm, about 25 mm, about 30 mm, about 35 mm, between about 15 mm and about 35 mm, between about 10 mm and about 30 mm, and any value in between those ranges listed, including endpoints In some embodiments, the width of the clamp 330 in the mesial-distal direction is about 5 mm, about 10 mm, about 15 mm, between about 5 mm and about 15 mm, and any value in between those ranges listed, including endpoints In some embodiments, the clamp 330 can have a thickness between about 0.2 mm, about 0.4 mm, about 0.6 mm, about 0.8 mm, about 1.0 mm, about 1.2 mm, about 1.4 mm, about 1.6 mm, about 1.8 mm, between about 0.2 mm and about 1.8 mm, between about 0.4 mm and about 1.6 mm, between about 0.6 mm and about 1.4 mm, and between about 0.8 mm and about 1.2 mm, and any values in between those ranges listed, including endpoints. In some examples, the thickness of the clamp 330 can affect parameters such as the amount the clamp 330 can open, the opening force, the clamping force, and the stresses inside the clamp, etc.

In some embodiments, the clamp 330 can comprise a variety of materials such as titanium alloys (e.g., TiAl6V4) or stainless steel (e.g., type 440C). In some examples, the clamp 330 materials are biocompatible while also having sufficient yield strength and ductility. In some embodiments, the clamp 330 can be manufactured using a traditional metal forming process (e.g., bending). In some examples, the clamp 330 can be manufactured using CNC machining. In some embodiments, the clamp 330 can be sterilized using an autoclave. In some examples, the clamp 330 can be made of a spring steel with a biocompatible metal coating (e.g., nickel plating), a tempered stainless steel (e.g., Type 440C), or titanium alloys (e.g., TiAl6V4). The clamp can then be sterilized using autoclaves.

As shown in FIGS. 4 and 5 of the sensor unit 310, the MSU 320 is configured to be received within the opening 336a and opening 336b of the clamp 330. In some embodiments, the MSU 320 has a “headphone design” to provide for easy assembly. In some examples, the MSU 320 is designed to be inserted into the clamp 330 to allow for clear line of sight of the target treatment area (e.g., the abutment or frown). In some embodiments, the MSU 320 can be positioned within a barrier sleeve during use to provide for simple infection control procedure and reuse of the MSU 320. In some embodiments, the MSU 320 can include a force generator 322, a sensor 324, and a clip 326. In some embodiments, the force generator 322 can be a motor such as a brushless DC motor or a linear resonant actuator that is configured to provide a haptic feedback. In some examples, the sensor 324 can be a digital MEMS accelerometer. In some embodiments, the force generator 322 and the sensor 324 are electrically connected. This can be done in any variety of methods such as, for example, a flexible printed circuit board (FPCB), a plurality of electrical wires, etc. In some embodiments, the force generator 322, the sensor 324, and the electrical connection is molded or encapsulated to form the inverted U-shape clip 326. In some embodiments, the molding or encapsulation can be done using a material such as a resin, polymer or plastic. In some embodiments, the MSU 320 is elastic. This can allow the MSU 320 to open with the clamp 330. In some embodiments the MSU 320 is preloaded to keep the MSU 320 on the clamp 330.

As shown in FIGS. 4 and 5, the force generator 322 is positioned such that it extends in a perpendicular direction relative to the lower portion 330b of the clamp 330. In some embodiments, the force generator 322 forms an inclination with the shorter claw of the pair of claw 332 and claw 334. In some embodiments, the inclination formed is about 5°, about 10°, about 15° about 20°, about 25°, about 30°, about 35°, about 40°, about 45°, between about 5° and about 45°, between about 10°, and about 40°, and any values in between those ranges listed, including endpoints. In some embodiments, the force generator 322 is positioned such that the plane of the force generator 322 passes through a center of the shorter claw (e.g., claw 332). This can allow the oscillating force generated by the force generator 322 to pass through a center of the claw 332. In some embodiments, this can ensure that once the clamp 330 is attached to the abutment or crown, the force generator 322 can act directly on the center of the abutment or crown. The position of the application of force can ensure that the force generator 322 does not create any moment to rock the clamp 330. This can help to eliminate excessive vibration that could deteriorate the signal-to-noise ratio of the sensor unit 310.

In some embodiments, the sensor 324 is positioned adjacent to the longer claw of the pair of claw 332 and claw 334. In some examples, the sensor 324 extends in a perpendicular direction with the longer claw (i.e., claw 334). In some embodiments, this configuration allows the sensor 324 to only measure the motion of the abutment or crown but cannot sense the rocking of the clamp 330. This can further improve the signal-to-noise ratio of the sensor unit 310 as measurement noise is minimized.

In some examples, the clip 326 is offset to one side such that it is not located at the mid-plane of the force generator 322 and the sensor 324. In some embodiments, this offset can allow the clip 326 to engage in the opening 336a and opening 336b on the clamp 330 when the MSU 320 is engaged with the clamp 330. In some examples, the opening 336a and the opening 336b are designed to secure the MSU 320 and prevent it from sliding up or down relative to the clamp 330. In some embodiments, the clip 326 of the MSU 320 can be planar or warped. When the MSU 320 is warped, the MSU 320 can have increased rigidity against twisting. In some embodiments, the MSU 320 can include a protrusion 328 that is positioned on a portion of the clip 326 adjacent to the force generator 322. The force generator 322 can provide a number of functions. In some embodiments, the protrusion 328 can serve as an axial stop. During deployment of the sensor unit 310 onto an abutment or crown, the protrusion 328 will first contact a top contour of the abutment or crown. This can provide a tactile feedback to a user that the sensor unit 310 has reached the target position and the user can release the clamp 330 to attach it to the abutment or crown. In some examples, the protrusion 328 along with the claw 332 and the claw 334 can form an attachment mechanism for holding the clamp 330 and MSU 320 onto the abutment. In some embodiments, the protrusion 328 can prevent downward movement of the clamp 330 during activation of the sensor unit 310. As the force generator 322 is activated during stability measurements, the clamp 330 may rock and cause the clamp 330 to “walk” down the abutment or crown. The protrusion 328 can help to prevent this movement.

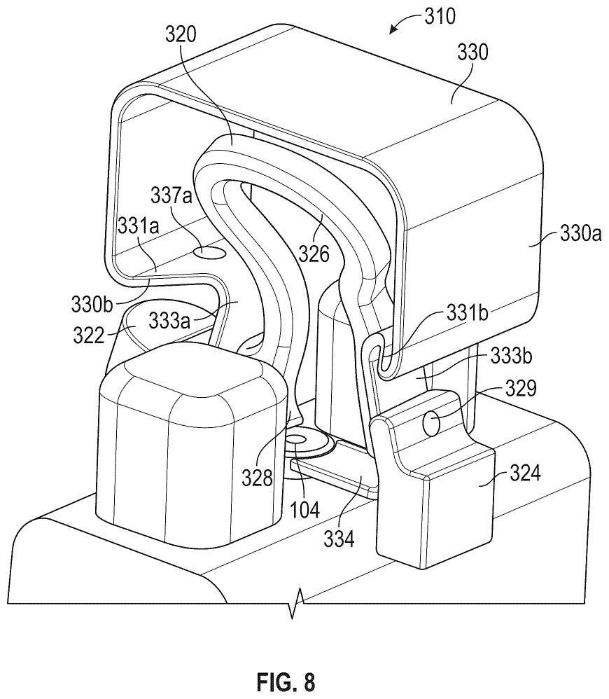

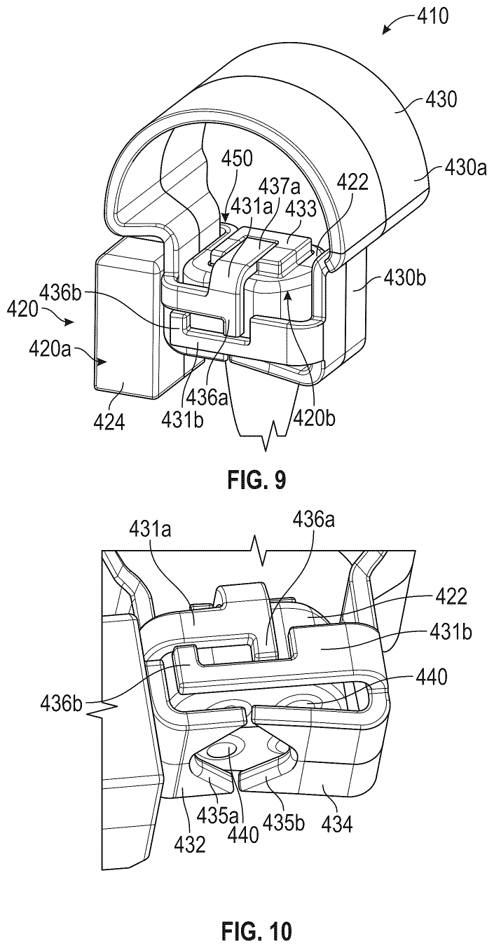

In some embodiments, an electrical cable can be provided to connect the 420 with a controller. In some examples the electrical cable can transfer power from the controller to drive the force generator 322 and sensor 324. In some embodiments, the electrical cable can simultaneously transfer measured data from the sensor 324 back to the controller. In some embodiments the electrical cable is connected to the MSU 320 on the side of the MSU 320 where the sensor 324 is positioned. Because the sensor 324 is positioned on the buccal side, positioning the electrical cable adjacent to the buccal side of the MSU 320 can ensure that the electrical cable does not interfere with the tongue and does not block the line of sight of the user. FIGS. 8 and 9 illustrate an example of the location of the electrical connection 329 on the portion of the MSU 320 that houses the sensor 324. In some embodiments, the orientation of the connection point of the electrical connection 329 is somewhere between the buccal direction and the vertical direction. This can ensure that when the electrical cable extends out of the electrical connection 329, it does not interfere with the tips of the forceps through the hole 337a and hole 337b of the lower portion 330b of the clamp 330. In some embodiments, this can also allow the electrical wire to turn toward the mesial direction and exit the patient's mouth along the buccal side. In some embodiments, the length of the electrical cable is the distance between the sensor unit 310 and an external controller. In some embodiments, the electrical cable has a length of about 12 inches, about 24 inches, about 36 inches, about 48 inches, about 60 inches, between about 12 inches to about 60 inches, between about 24 inches to about 48 inches and any values in between those ranges listed, including endpoints.

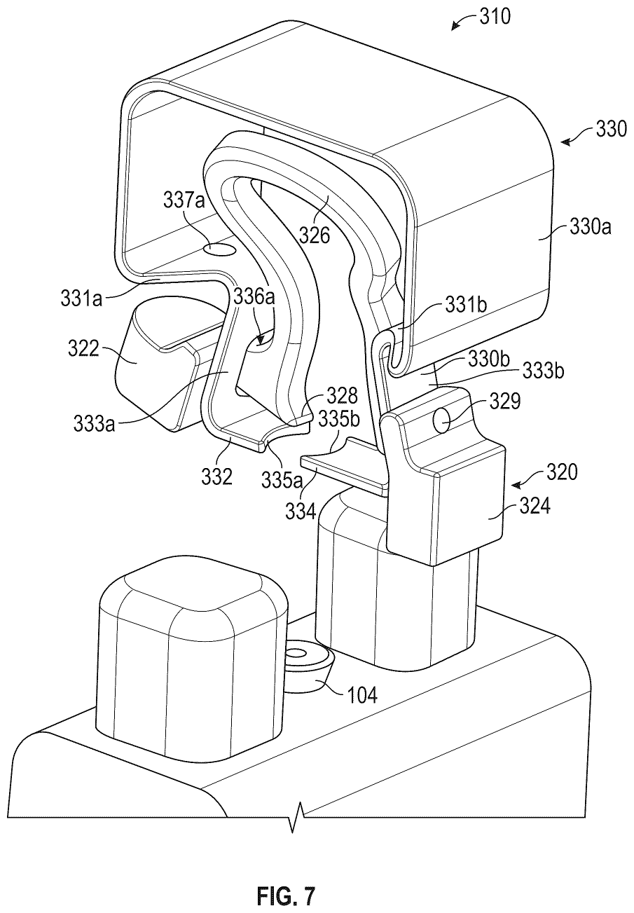

The disclosed sensor unit 310 is designed to be used on all teeth regardless of size or position (e.g., left side or right side of the mandible or maxilla). FIG. 7 illustrates an embodiment of the sensor unit 310 before it engages an abutment (shown) or crown (not shown). FIG. 8 illustrates an embodiment of the sensor unit 310 once it is engaged with the abutment (shown) or crown (not shown). To deploy the sensor unit 310, the MSU 320 with its electrical cable (not shown) is first positioned within a disposable barrier sleeve. This can allow the MSU 320 to be reused. The MSU 320 can then secured onto the clamp 330 through the opening 336a and opening 336b on the lower portion 330b. In some embodiments, if additional anti-slip capabilities are required, the MSU 320 can be warped to increase the twisting rigidity. This can provide a twisting moment that serves as an extra protection to prevent the clamp 330 from disengaging from the clamp 330. This can help to hold the MSU 320 onto the clamp. A pair of forceps can then be used to open the clamp 330 to place the MSU 320 onto the abutment or crown. As shown in FIG. 8, once the forceps are released, the claws 332, 334 of the clamp 330 and the protrusion 328 of the MSU 320 can firmly anchor the sensor unit 310 to the top 1-2 mm of the abutment or crown. In some embodiments, the motor can then be turned on and an oscillating force applied to the abutment or crown. In some embodiments, the oscillating force is about gum line 100 Hz, about 150 Hz, about 200 Hz, about 250 Hz, about 300 Hz, about 350 Hz, about 400 Hz, about 450 Hz, about 500 Hz, about 550 Hz, about 600 Hz, about 650 Hz, about 700 Hz, about 750 Hz, about 800 Hz, about 850 Hz, about 900 Hz, about 950 Hz, about 1 kHz, between about 100 Hz to about 1000 kHz, between about 100 Hz to about 550 Hz, between about 150 Hz to about 500 Hz, about 150 Hz to about 450 Hz, between about 200 Hz to about 400 Hz, between about 250 Hz to about 350 Hz, and any values in between those ranges listed, including endpoints. In some examples, the sensor 324 then measures the abutment or crown's response to the oscillation of the sensor 324. In some embodiments, the measured forced force and acceleration are processed to obtain implant stability. As shown in FIG. 8, the abutment can still be seen through the sensor unit 310, illustrating that the sensor unit 310 can ensure that the line of sight is not blocked during the measurement of implant stability. In some embodiments, once the measurements are completed, the MSU 320 is released from the abutment or crown. In some embodiments, this is done using forceps. The clamp 330 and the MSU 320 can be disassembled. In some embodiments, the clamp 330 can be autoclaved for sterilization. In some examples, the barrier sleeve can be removed from the MSU 320 and disposed. The MSU 320 can then be cleaned with a disinfectant wipe for reuse.

FIGS. 9 and 10 illustrate another embodiment of a sensor unit 410. The sensor unit 410 resembles or is identical to the system sensor unit 310 in many respects. Accordingly, numerals used to identify components of the system for sensor unit 310 are incremented by a factor of one hundred to identify like features of the system for sensor unit 410. This numbering convention generally applies to the remainder of the figures. Any component or step disclosed in any embodiment in this specification can be used in other embodiments.

In some embodiments, the sensor unit 410 includes a MSU 420 and a clamp 430. As shown in FIG. 9, the clamp 430 can have an inverted U-shape. In some embodiments, the clamp 430 can have an upper portion 430a and a lower portion 430b. In some examples, the upper portion 430a can have a curved arch and the lower portion 430b can have a straight walls. In some embodiments, the MSU 420 comprises a first portion 420a and a second portion 420b. The first portion 420a can be in the form of a block and the second portion 420b can be in the form of a deep slot 450. In some embodiments, a force generator 422 is positioned in the first portion 420a of the MSU 420. As discussed above, in some embodiments, the force generator 422 is a motor. The first portion 420a can be located inside the clamp 330. The force generator 422 can be positioned either vertically or horizontally. For example, as shown in FIG. 10, the force generator 422 is positioned horizontally in the clamp 330. In some embodiments, a sensor 424 can be positioned within the second portion 420b. In some examples, the sensor 424 can be an accelerometer. The second portion 420b can be located on the outside of the clamp 330. In some embodiments, a width of the deep slot 450 is slightly smaller than a thickness of the lower portion 450b of the clamp 330 which can allow an interference fit between the MSU 420 and the clamp 430. In some embodiments, the MSU 420 and the clamp 430 are designed such that when the MSU 420 slides onto the clamp 430 through the deep slot 450, the interference fit generates friction to connect the clamp 430 and MSU 420 together to form the sensor unit 410.

In some embodiments, the sensor unit 410 includes a pair of anti-yield hooks (hook 431a and hook 431b) positioned on the side of the clamp 430. The anti-yield hooks 431a, 431b are designed to prevent damage to the clamp 430. A user using a pair of forceps to open the clamp 430 can unconsciously apply an excessive grip force. Such a force could cause the clamp 430 to open too side and cause the material of the clamp 430 to yield. As can be seen in FIGS. 9 and 10, the end 436a of the hook 431a and the end 436b of the end 436a are designed to limit the amount the clamp 430 can open.

In some embodiments, interference between the MSU 420 slot (i.e. first portion 420a) and the clamp thickness can be reduced by engaging a guide tab 437a on the clamp 430 and a U-shaped lock 433 on the MSU 420. In some embodiments, a large interference can produce a significant friction force between the clamp 430 and the MSU 420 to keep each of the clamp 430 and the MSU 420 in place on the sensor unit 410. However, this large interference can also create tremendous stress in a portion of the MSU 420 that bridges the first portion 420a of the MSU 420 and the second portion 420b of the MSU 420. To alleviate the stresses in the portion of the MSU 420 that bridges the first portion 420a and the second portion 420b, the guide tab 437a on the clamp 430 is designed to retain the MSU 420 on the U-shaped lock 433. In some embodiments, a smaller interference can therefore be used because the guide tab 437a and the U-shaped lock 433 will assist in retaining the connection between the MSU 420 and clamp 430.

FIG. 10 illustrates an embodiment of the attachment mechanism of the sensor unit 410. The sensor unit 410 can include a claw 432 and a claw 434 with a engagement portion 435a and engagement portion 435b respectively for engaging the abutment or crown. In some embodiments the engagement portion 435a of the claw 432 and the engagement portion 435b of the claw 434 have an inverted U-shape. In some embodiments, as shown in FIG. 10, the MSU 420 has a plurality of bumps 440. In some embodiments, the bumps 440 on the MSU 420 can serve as an axial stop. In some examples, then the clamp 430 is deployed onto an abutment or crown, each of the bumps 440 can first contact a top surface of the abutment or crown. This can indicate to a user that the MSU 420 is in position. In some embodiments, a user can release the forceps used to open the sensor unit 410, and the clamp 430 will close with the clamp 430 firmly engaged with the abutment or crown. In some embodiments, the claws (claw 432, claw 434) on the clamp and the plurality of bumps 440 on the MSU 420 collectively form an attachment mechanism to anchor the sensor unit 410 to the abutment or crown.

As shown in FIGS. 9 and 10, in the sensor unit 410, the force generator 422 is located inside the clamp 430. In the configuration shown, the position of the force generator 422 ensures that the sensor unit 410 does not interfere with the tongue. In some embodiments, because the force generator 422 is positioned above the abutment or crown, the force generator 422 can generate a force that may produce unwanted vibration. In some examples, the sensor 424 is therefore positioned near the claw 432 and claw 434 to filter out measured noise resulting from any unwanted vibration.

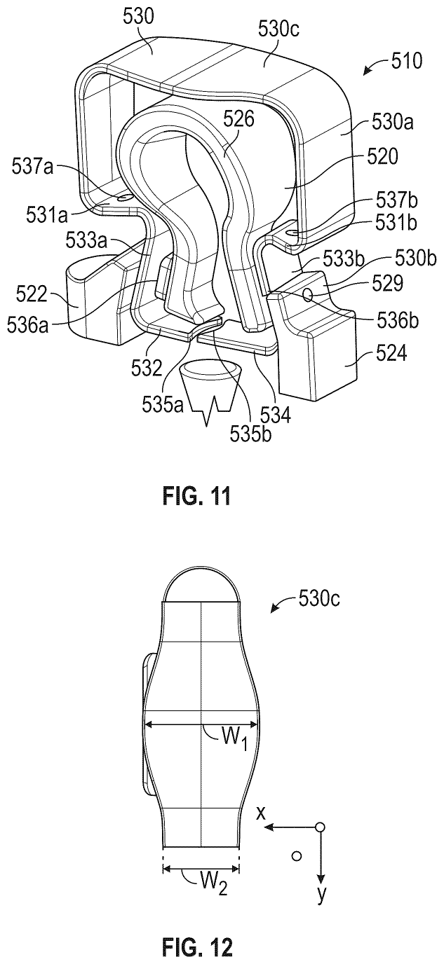

FIG. 11 illustrates another embodiment of a sensor unit 510. The sensor unit 510 resembles or is identical to the system sensor unit 310 in many respects. Accordingly, numerals used to identify components of the system for sensor unit 310 are incremented by a factor of one hundred to identify like features of the system for sensor unit 510. This numbering convention generally applies to the remainder of the figures. Any component or step disclosed in any embodiment in this specification can be used in other embodiments.

In some embodiments, the sensor unit 510 includes MSU 520 and a clamp 530. In some embodiments the clamp 330 includes an upper portion 530a and a lower portion 530b. In some examples, the 530b includes a first portion 531a, 531b, a second portion 533a, 533b, and claws (claw 532 and claw 534). In some embodiments, the second portion 533a and the second portion 533b can include an opening 536a and an opening 536b respectively that are configured to receive and retain the MSU 320. In some examples, the first portion 531a and the first portion 531b include a hole 537a and a hole 537b respectively that are configured to receive a forceps for opening the sensor unit 510. In some embodiments, the MSU 520 includes a force generator 522 and a sensor 524 that are electrically connected by a clip 526.

In some embodiments, an electrical cable can be provided to connect the 520 with a controller. In some examples the electrical cable can transfer power from the controller to drive the force generator 522 and sensor 524. In some embodiments, the electrical cable can simultaneously transfer measured data from the sensor 524 back to the controller. In some embodiments the electrical cable is connected to the MSU 520 on the side of the MSU 520 where the sensor 524 is positioned. Because the sensor 524 is positioned on the buccal side, positioning the electrical cable adjacent to the buccal side of the MSU 520 can ensure that the electrical cable does not interfere with the tongue and does not block the line of sight of the user. FIG. 11 illustrates an example of the location of the electrical connection 529 on the portion of the MSU 520 that houses the sensor 524. In some embodiments, the orientation of the connection point of the electrical connection 529 is somewhere between the buccal direction and the vertical direction. This can ensure that when the electrical cable extends out of the electrical connection 529, it does not interfere with the tips of the forceps through the hole 537a and hole 537b of the lower portion 530b of the clamp 530. In some embodiments, this can also allow the electrical wire to turn toward the mesial direction and exit the patient's mouth along the buccal side. In some embodiments, the length of the electrical cable is the distance between the sensor unit 510 and an external controller. In some embodiments, the electrical cable has a length of about 12 inches, about 24 inches, about 36 inches, about 48 inches, about 60 inches, between about 12 inches to about 60 inches, between about 24 inches to about 48 inches and any values in between those ranges listed, including endpoints.

In some embodiments, the clamp 530 has a top surface 530c that has a curved profile. FIG. 12 illustrates a top view of the top surface 530c. In some embodiments, the top surface 530c has a central portion with a width w₁ that is about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, between about 6 mm and about 18 mm, between about 7 m and about 17 mm, between about 8 mm and about 16 mm, between about 9 mm and about 15 mm, between about 10 mm and about 14 mm, between about 11 mm and about 13 mm, and any values in between those ranges listed, including endpoints. In some embodiments, the top surface 530c has end portion with a width w₂ that is about 5 mm, about 6 mm, about 7 mm, about 8 mm, about 9 mm, about 10 mm, about 11 mm, between about 5 mm and about 11 mm, between about 6 mm and about 10 mm, between about 7 mm and about 9 mm, any values in between those ranges listed, including endpoints. In some examples, the top surface 530c is gradually curved from the central portion to each of the end portions such that the sides are connected to the vertical walls of the clamp 530.

In some embodiments, the sensor unit 510 is deployed using a pair of forceps. In some examples, the tips of the forceps engage the hole 537a and the hole 537b on the clamp 530 to open the sensor unit 510. In some embodiments, forceps have a curved portion adjacent to where the tips are located. When the tips of the forceps go into the holes, the curved section of the forceps can interfere with the top surface 530c of the clamp 530. The curved profile of the clamp 530 creates a space for the curved section of the forceps. This can help to eliminate potential interference between the forceps and the clamp 530. In some embodiments, the curved profile of the clamp 530 can allow the vertical walls of the upper portion 530a to be further reduced. This can minimize the likelihood of the sensor unit 510 interfering with the mating teeth, particularly at the molar positions.

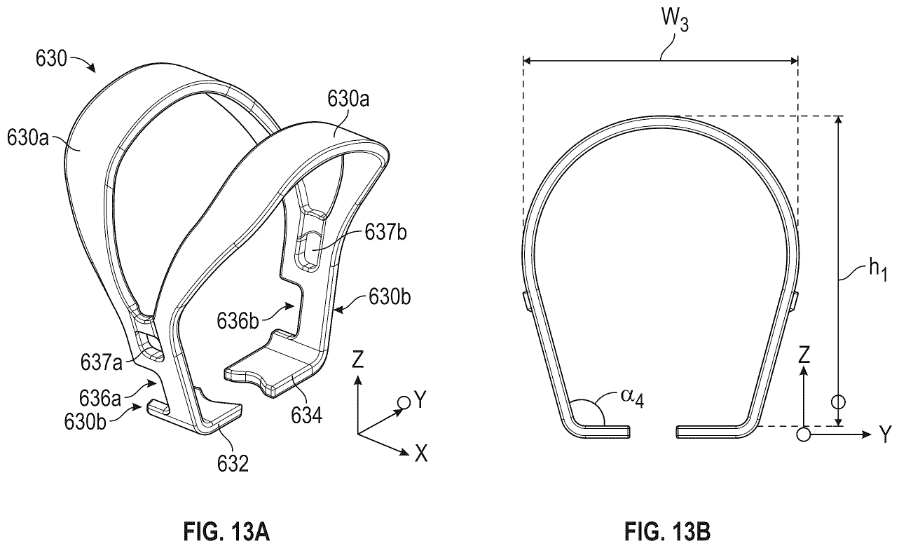

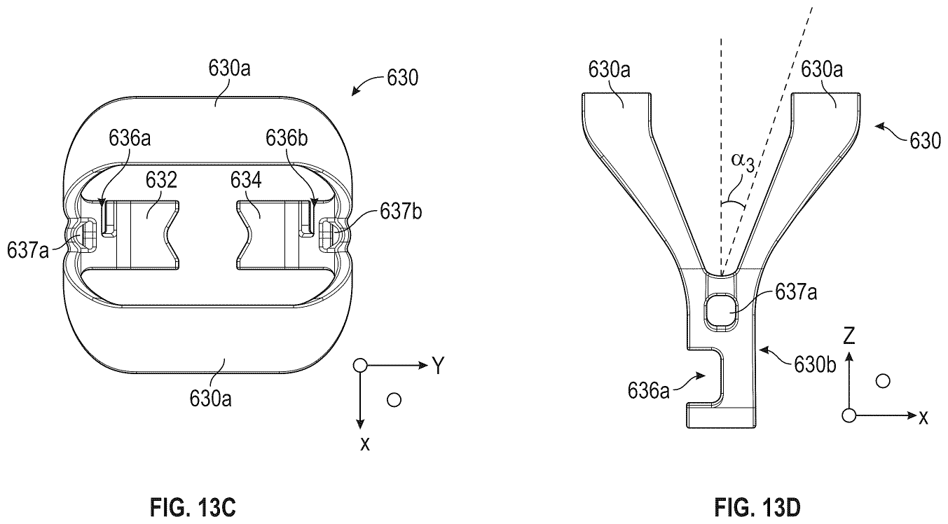

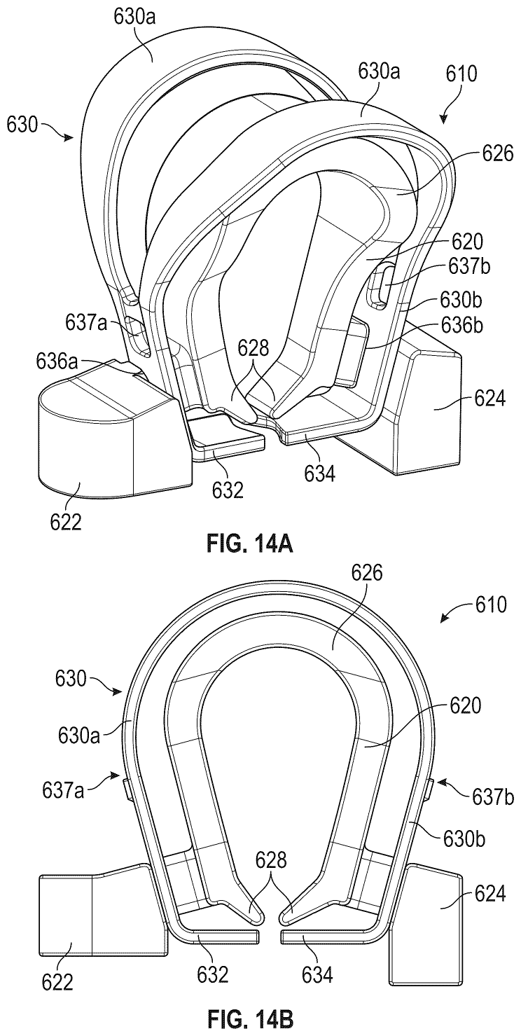

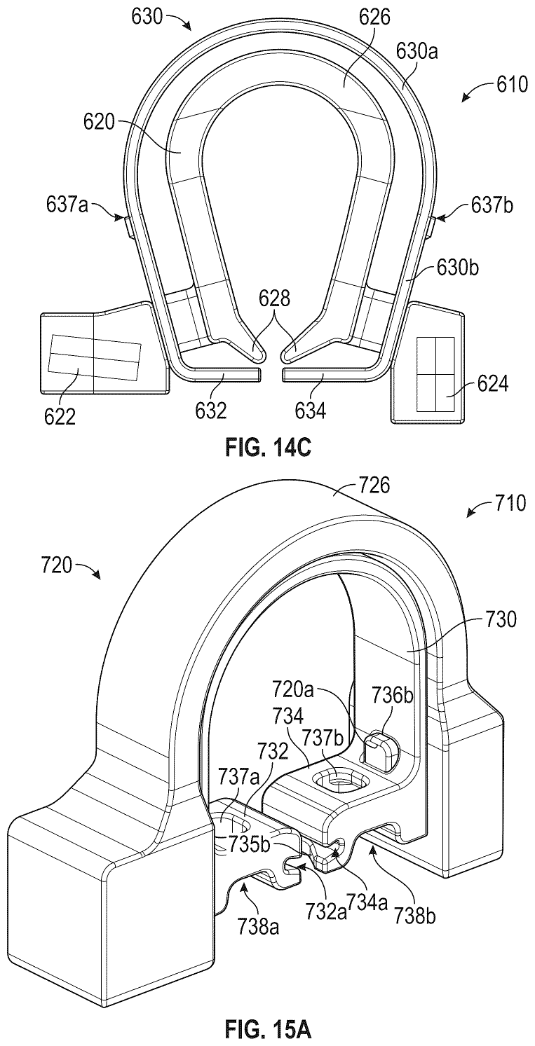

FIGS. 14A-14C illustrate an embodiment of a sensor unit 610 with a clamp 630 with reduced bends. FIGS. 13A-13D illustrate the clamp 630 for use in the sensor unit 610. The clamp 630 of the sensor unit 610 has a reduced number of bends which can provide for simplified manufacturing. In some embodiments, structures with increased bends can require additional metal forming work. This can result in additional sources of dimensional inaccuracies. The metal forming process can be particularly challenging for materials such as titanium alloys as they are very ductile and can experience bounce-backs in manufacturing. MSDF.

FIGS. 13A-13D illustrates the clamp 630 of the sensor unit 610. The clamp 630 includes an upper portion 630a and a lower portion 630b. As shown, the 630 only has three bends. In some embodiments, a first bend formed by the circular arch of the upper portion 630a and a second and third bend at the bottom of each side of the lower portion 630b adjacent the claw 632 and the claw 634. In some embodiments, the second and third bends have an angle α₄ that is about 75°, about 80°, about 85°, about 90°, about 95°, about 100°, about 105°, about 110°, about 115°, about 120°, about 125°, about 130°, about 135°, between about 75° and about 135°, between about 80° and about 130°, between about 85° and about 125°, between about 90° and about 120°, between about 95° and about 115°, between about 100° and about 110°, and any values in between those ranges listed, including endpoints. In some embodiments, the lower portion 630b of the clamp 630 include a hole 637a and a hole 637b that are designed to receive a pair of forceps. In some embodiments, the upper portion 630a of the clamp 630 is divided into two separate wings that are spaced apart by an open strip. In some embodiments each of the wings forms an angle α₃ with a centerline of the clamp 630. In some examples, the angle α₄ is about 10°, about 11°, about 12°, about 13°, about 14°, about 15°, about 16°, about 17°, about 17.05°, about 17.10°, about 17.15°, about 17.20°, about 17.25°, about 17.30°, about 17.35°, about 17.40°, about 17.45°, about 17.50°, about 17.55°, about 17.60°, about 17.65°, about 17.70°, about 17.75°, about 17.80°, about 17.85°, about 17.90°, about 17.95°, about 18°, about 19°, about 20°, about 21°, about 22°, about 23°, about 24°, about 25°, between about 10° and about 25°, between about 11° and about 24°, between about 12° and about 23°, between about 13° and about 22°, between about 14° and about 21°, between about 15° and about 20°, between about 16° and about 19°, and any values in between those ranges listed, including endpoints. In some embodiments the two wings and open strip of the upper portion 630a of the clamp 630 can form a large opening that allow a pair of forceps tips to enter the clamp 630 to engage the forceps holes (hole 637a and hole 637b). In some embodiments, the clamp 630 can have additional bends. For example, the clamp 630 can have 4 bends, 5 bends, 6 bends, 7 bends, 8 bends, 9 bends, or more bends.

In some embodiments, the clamp 630 can include an opening 636a on a first side of the lower portion 630b and an opening 636b on a second side of the lower portion 630b. The opening 636a and the opening 636b can have dimensions that allow the MSU 620 to be inserted and retained by the clamp 630.

FIGS. 14A-14C illustrate an embodiment of the MSU 620 that includes a force generator 622 on a first side of the MSU 620 and a sensor 624 on a second side of the MSU 620. In some embodiments the force generator 622 is a motor. FIG. 14C illustrates a cross-sectional view of the MSU 620 and illustrate the position of the force generator 622 and the sensor 624 within the MSU 620. In some examples, the sensor 624 is an accelerometer. In some embodiments, the force generator 622 is slightly tilted within the MSU 620. This can allow the force generator 622 to direct a force through the center of the claws 632,634 and through a center of the abutment or crown. In some embodiments, the force generator 622 and the sensor 624 are electrically connected through the clip 626 extending between the force generator 622 and the sensor 624. In some embodiments, the MSU 620 can include a pair of protrusions 628 that serve as axial stops on the MSU 620. In some embodiments, the MSU 620 may include at least one protrusion 628 that radially extends inward. In some examples, the protrusion 628 may extend more radially inward than an adjacent claw. In some examples, the protrusion 628 may be configured to abut a top of the abutment or crown and prevent the sensor unit 610 from sliding down the abutment or crown during use.

In some embodiments the MSU 620 can be preloaded to allow the MSU 620 to more easily attach onto the clamp 630. In some examples, the MSU 620 may be attached to the clamp 630 by sliding the MSU 620 into the clamp 630 from a lateral position. In some embodiments, the MSU 620 may slide between the wings of the upper portion 630a of the clamp 630.

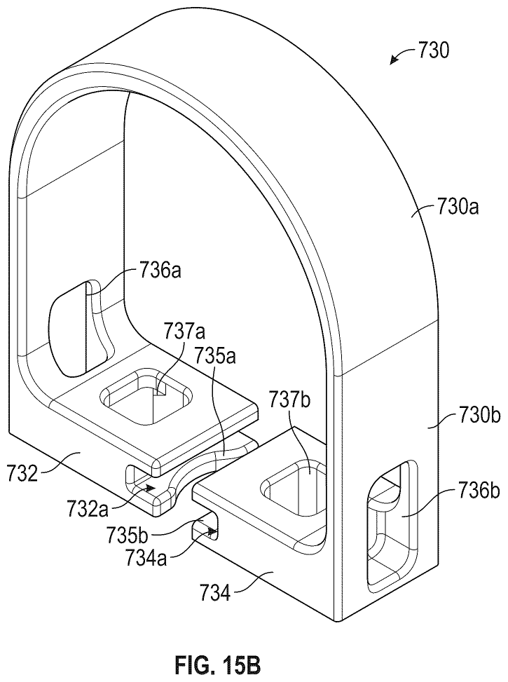

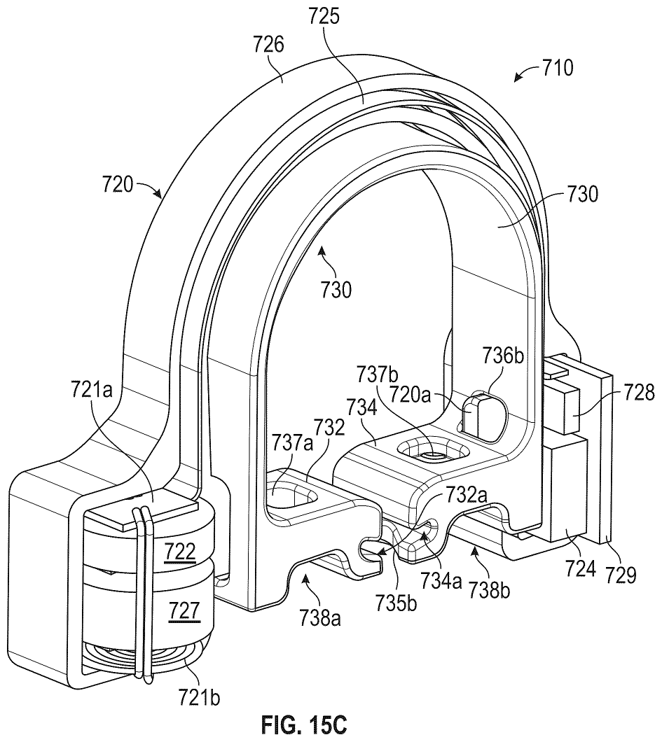

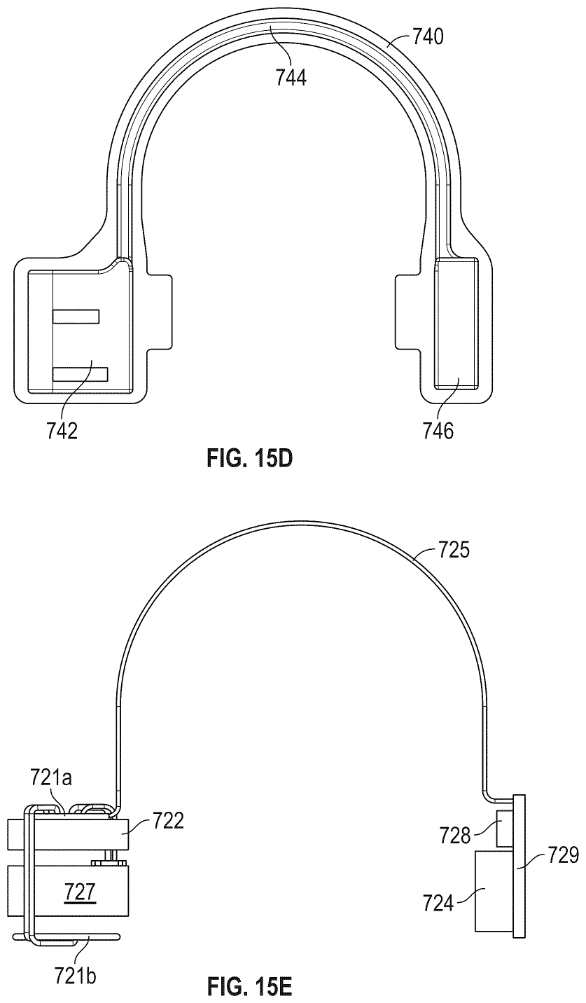

FIGS. 15A-15E illustrate an embodiment of a sensor unit 710 that is wireless. The sensor unit 710 resembles or is identical to the system sensor unit 310 in many respects. Accordingly, numerals used to identify components of the system for sensor unit 310 are incremented by a factor of one hundred to identify like features of the system for sensor unit 710. This numbering convention generally applies to the remainder of the figures. Any component or step disclosed in any embodiment in this specification can be used in other embodiments. In some embodiments, the sensor unit 710 includes a MSU 720 and a clamp 730. In some examples, the MSU 720 has wireless capabilities and there is no electrical cable connecting the MSU 720 with an external controller. In some embodiments, the MSU 720 can rely on a wireless connection (e.g., Bluetooth) to transmit a measured acceleration data to an external application (e.g., mobile application). In some embodiments, the controller is incorporated in the MSU 720.

FIG. 15A illustrates a sensor unit 710 with clamp 730 and a wireless MSU 720. As shown, in some embodiments, the clamp 730 is positioned on the inside of the sensor unit 710. In some examples, the MSU 720 is attached onto an outside surface of the clamp 730 like a headphone worn on a head. In some embodiments, the clamp 730 has an upper portion 730a and a lower portion 730b. In some examples, the upper portion 730a has an inverted U-shape. In some embodiments, the lower portion 730b comprises a pair of holes 737a, 737b, a pair of openings 736a,736b, and a pair of claws 732, 734.

In some examples, the lower portion 730b includes a hole 737a and a hole 737b through which a pair of forceps can be inserted to open the clamp 730 so that the sensor unit 710 can be attached to a healing abutment or a crown. In some embodiments, because the hole 737a and the hole 737b are positioned at the lower portion 730b of the clamp 730, the increased distance between the holes 737a, 737b and an apex of the upper portion 730a can form a large moment arm. This can reduce the forces required to open the clamp 730.

In some embodiments the clamp 730 has a non-uniform thickness. For example the inverted U-shape portion of the upper portion 730a can have a thinner but uniform thickness, while the lower portion 730b can have a larger and non-uniform thickness with an undercut 738a and undercut 738b positioned on either side of the clamp 730. In some embodiments, the undercut 738a and the undercut 738b can allow the claws 732, 734 to be positioned between the abutment/crown and its surrounding gingiva.

In some embodiments, the increased thickness of the lower portion 730b can enhance clamping effectiveness onto an abutment or crown. In some embodiments, each of the claws 732, 734 include an axial stop 728a, 728b, an engagement portion 735a, 735b with two inclined straight edges, and a recess 732a, 734a. In some examples, the axial stop 728a and axial stop 728b are in the form of a flat surface. The axial stop 728a and axial stop 728b can be configured to ensure that the clamp 730 stays on the abutment or crown at a designated position. The axial stop 728a and axial stop 728b can also provide axial support when the clamp 730 is attached onto the abutment or crown. The inclined straight edges of each of the engagement portion 735a and engagement portion 735b can provide lateral clamping forces and lateral support when the clamp 730 is attached onto an abutment or crown. In some embodiments, the purpose of the recess 732a and recess 734a is to ensure that each of the claw 732 and claw 734 can attach onto any abutment or crown of arbitrary geometry. For example, a commonly used abutment can have an inverted conical shape which can be difficult to attach. The recess 732a and recess 734a can provide the claw 732 and claw 734 with lateral space such that the axial stop 728a and axial stop 728b and the straight edges of the engagement portion 735a and engagement portion 735b will simultaneously attach onto the abutment. In some embodiments, the recess 732a and recess 734a, can ensure that the clamp 730 and abutment contacts at the axial stops 728a, 728b and engagement portions 735a, 735b regardless of the geometry of the abutment or crown. In some embodiments, if the abutment is slightly taller than the gingiva, a clamp 730 without an under cut can be used. An example of this is shown in FIG. 15B.

In some embodiments, the MSU 720 and the clamp 730 are assembled through a clipping engagement. In some embodiments, the MSU 720 can have a pair of protrusions 720a that are configured to fit into the opening 736a and opening 736b respectively. In some embodiments, the protrusions 720a are slightly larger than the opening 736a and opening 736b. This can form an interference fit between the MSU 720 and the clamp 730. In some examples, when the MSU 720 is opened and clipped onto the clamp 730, an elastic restoring force can be developed inside the MSU 720 to hold the MSU 720 onto the clamp 730. In some embodiments, the interference fit between the protrusions 720a and the MSU 720 can increase measurement accuracy by ensuring that the MSU 720 moves with the clamp 730.

In some embodiments, the clamp 730 can comprise a titanium alloy. The properties of titanium alloy (e.g., high Young's modulus) can allow the clamp 730 to have a large clamping force while having enough flexibility to open the clamp 730 for a wide range of abutment/crown diameters. In some embodiments, the clamp 730 can be made of stainless steel after suitable heat treatments. In some embodiments, the clamp 730 can be manufactured via CNC machining or wire electrical discharge machining.

FIG. 15C illustrates a cross-sectional view of the MSU 720. The MSU 720 can include MSU housing 740 and a plurality of electronics housed within the MSU housing 740. FIG. 15E illustrates the electronics housed within the MSU housing 740. In some embodiments, the electronics can include a force generator 722 (e.g., a motor), a battery 727, a charging circuit 721a and a charging coil 721b, a sensor 724 (e.g., an accelerometer), and a Bluetooth chipset 728 on a PCB 729. In some embodiments, the force generator 722, the battery 727, and the charging circuit 721a with the charging coil 721b are positioned on a first side of the MSU 720. In some embodiments, the sensor 724, the Bluetooth chipset 728, and the PCB 729 are positioned on a second side of the MSU 720. In some embodiments, the electronics in the MSU 720 are connected by a flexible printed circuit board (PCB) 725. In some embodiments the force generator 722 can be a brushless DC haptic motor with a diameter of 6 mm and a heigh of 3.5 mm. In some examples, the battery 727 can be a lithium-ion button battery. In some embodiments, the charging circuit 721a and the charging coil 721b can be configured to support wireless charging. In some examples, the battery 727 can be charged through contact charging through a physical port. In some embodiments, the Bluetooth chipset 728 can enable the wireless transmission of the measured data from the sensor 724 to an external application (e.g., mobile application). In some embodiments, the Bluetooth chipset 728 can have a small footprint. For example, it can have a dimension of 4 mm by 4 mm. In some embodiments, the sensor 724 is a digital triaxial accelerometer with a dimension of 3 mm by 3 mm.

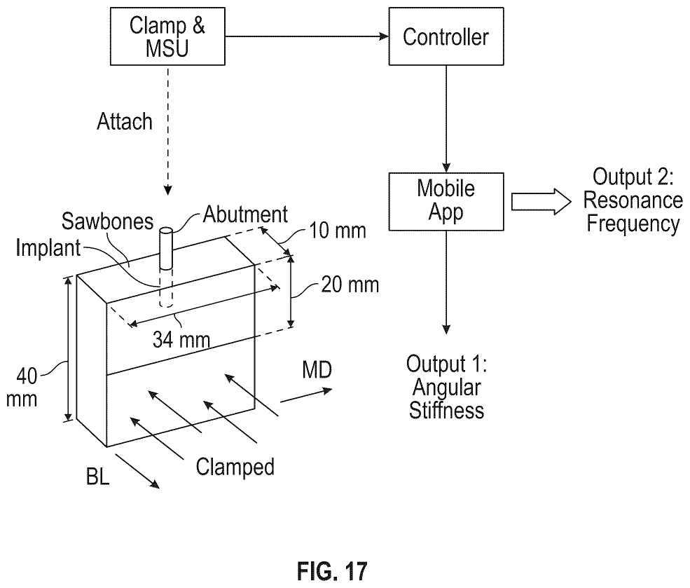

FIG. 17 17D illustrates a cross-sectional view of the MSU housing 740. In some embodiments, the MSU housing 740 includes a cavity 742, a curved slot 744, and a cavity 746. In some embodiments, the cavity 742 is designed to receive the force generator 722 and the battery 727. In some embodiments, the cavity 746 is designed to received the sensor 724 and the Bluetooth chipset 728. In some examples, the slot 744 can receive the flexible PCB 725.

As mentioned previously, except as otherwise disclosed herein, the dental implant stability analyzers disclosed therein may be the same as any of the other dental implant stability analyzers disclosed herein. For example, any of the dental implant stability analyzers may exhibit any of the dimensions, angle, etc. disclosed herein, without limitation.

### Method of Manufacturing

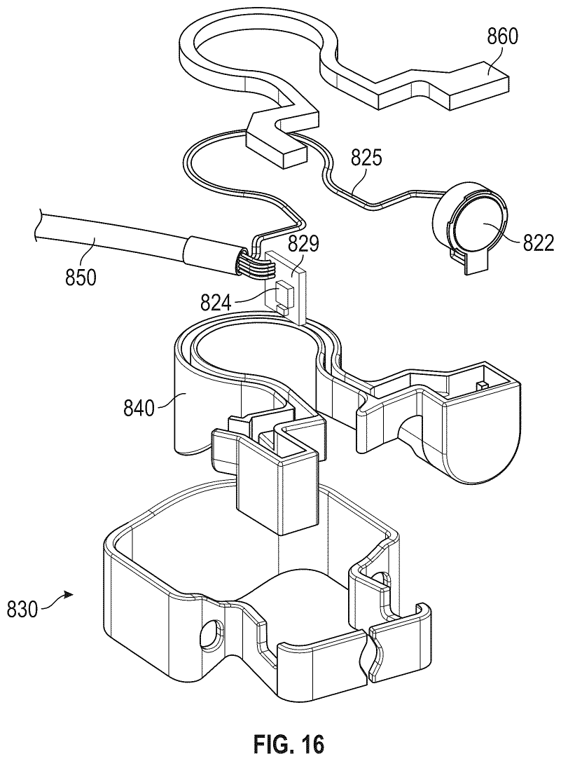

FIG. 16 illustrates an exploded view of the sensor unit 810 to illustrate the manufacturing and assembly of a wired sensor unit 810. In some embodiments, the MSU 820 is first machined via CNC or through injection molding. In some embodiments, the material of the MSU housing 840 can be Nylon 66 or PEEK. As discussed above, the MSU housing 840 can include a continuous cavity to receive the electronics subassembly.

In some embodiments, the electronics subassembly can be formed as follows. In some examples, the sensor 824 (e.g., accelerometer) can be surface mounted to a rigid printed circuit board (PCB) 829 with a bypass capacitor to filter out high-frequency noise. In some embodiments, a force generator 822 (e.g., haptic motor) can be electrically connected to the PCB 829 via the flexible PCB 825. In some embodiments the connection between the force generator 822 and the PCB 829 is through two electrical wires of a length to match the length of the cavity of the MSU housing 840. In some embodiments, an electrical cable 850 with a plurality of electrical wires (e.g., 6 strands) is then soldered to the PCB 829. The electrical cable 850 can be shielded with thermoplastic elastomer with strain relief near the solder joints where the electrical cable 850 exits the MSU housing 840. In some embodiments, the electronics subassembly is then secured into the cavity of the MSU housing 840.

In some embodiments, a potting compound 860 is filled within the MSU housing 840 to encapsulate the electronics subassembly therein. In some embodiments, the potting compound 860 can be a medical grade epoxy. In some examples, if the potting compound 860 completely encapsulate the electronics subassembly, the manufacturing and assembly of the MSU 80 is completed. In some embodiments, the potting compound 860 does not encapsulate the electronics subassembly, an upper MSU cover (not shown) may need to be attached onto the MSU housing 840 to fully encapsulate the electronics subassembly. In some embodiments, the aforementioned manufacturing process can be applied to a wireless sensor unit.

### Testing and Calibration

In some embodiments, the accuracy of the clamp and the MSU system in an implant stability analyzer can be evaluated in a laboratory environment using a standardized specimen with a standardized test procedure. FIG. 17 illustrates an embodiment of a standardized specimen measured by a clamp and MSU to generate an angular stiffness value to indicate implant stability. In some embodiments, the standardized specimen is designed to mimic a premolar section of mandible. In some examples, the standardized specimen has a rectangular shape in the form of a slab. The slab can have dimensions of, for example, a length of 34 mm, a thickness of 10 mm, and a height of 40 mm. The thickness direction can mimic the buccolingual (BL) direction and the length direction can mimic the mesiodistal (MD) direction. In some embodiments, the lower half of the specimen in height is secured in a vise to provide a stable foundation for the specimen. In some embodiments, the material of the standardized specimen can be any material that mimics bone structures. For example, the material of the standardized specimen can be artificial bones (e.g., sawbones) or acrylic. In some embodiments, the material can be uniform throughout the specimen. In some examples, the material of the specimen can be composite. For example, a uniform Sawbones block of one density (e.g., 20 pounds per cubic foot, aka PCF) can serve as a base block, and a layer of a uniform Sawbones sheet of a second density (e.g., 2-mm thick layer with 40 PCF) can be laminated on the base block to form a composite specimen.

In some embodiments, at the center of the top face (i.e., the 34 mm×10 mm area) of the standard specimen, an implant can be inserted into the standardize specimen using an implantation protocol. In some examples, the protocol may be provided by the manufacturer of the implant. In some embodiments, the protocol may be created by the medical professional to mimic special clinical conditions (e.g., a smaller drill hole to increase traction and insertion torque for low density bones). Once the implant is inserted, an abutment can be attached onto the implant for the clamp and MSU to attach thereon. In some embodiments, the abutment can be a healing abutment, a locator abutment, a multi-unit abutment (MUA), or a custom abutment.

In some examples, once the standardized specimen is prepared, an implant stability analyzer with a clamp and MSU can be attached onto the abutment. In some embodiments, the implant stability analyzer can proceed to measure implant stability of the standardized specimen in the form of angular stiffness. In some embodiments, the value of the measured angular stiffness can vary depending on the density of the specimen, implant geometry/design, implantation protocol, etc. to reflect the implant stability. Although the angular stiffness is the main output to indicate implant stability, an implant stability analyzer can often implicitly extract resonance frequency as a byproduct from its measurements.

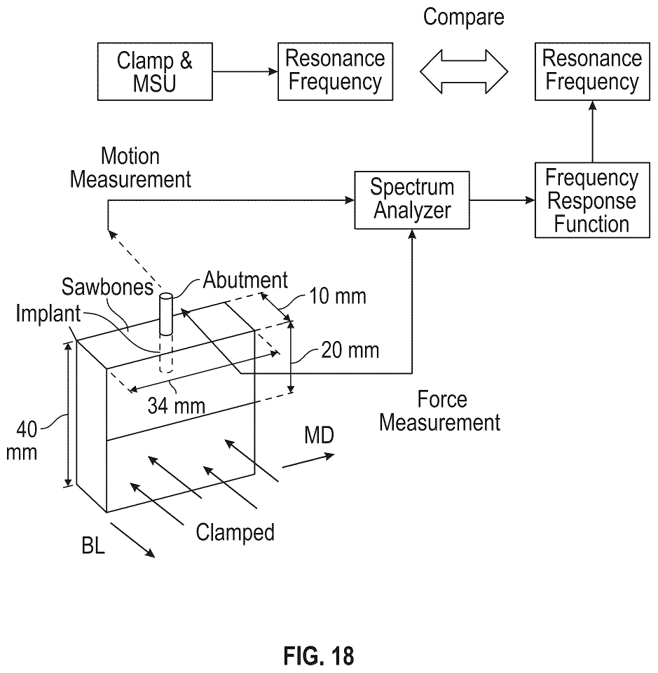

FIG. 18 illustrates an example of a standardized test procedure to calibrate an implant stability analyzer through the use of resonance frequencies. Resonance frequencies can be measured independently (without reference to an implant stability analyzer) in laboratory environments through use of a procedure called “experimental modal analysis.” In an “experimental modal analysis,” a force is applied, for example, through an impact hammer with a load cell to measure the force. A motion sensor can then be used to detect motion. In some embodiments, the motion sensor may be non-contact, such as a capacitance probe to measure displacement or a laser Doppler vibrometer to measure velocity. In some examples, the motion sensor may be in contact with the implant or abutment (e.g., use of an accelerometer). The measured force and motion can then be fed into a spectrum analyzer to obtain a frequency response function, from which a resonance frequency can be extracted.

In some embodiments, if an impact force is applied (e.g., using a stick to tap), the measured motion itself with a spectrum analyzer will be enough to independently identify the resonance frequency through an auto-spectrum of the measured motion. The spectrum analyzer can be replaced by a data acquisition system with software capable of performing Fourier analyses to convert the motion data measured in the time domain to its Fourier transform in the frequency domain, where a resonance frequency can be identified.

In some embodiments, once the resonance frequency is obtained from the experimental modal analysis, the resonance frequency can be compared with that obtained from the implant stability analyzer. In some examples, if a difference is detected, the algorithm can be adjusted in the implant stability analyzer so that the difference is minimized or kept in a range. The difference can define the accuracy of the implant stability analyzer. For example, a 2% difference in resonance frequency can mean 98% accuracy of the implant stability analyzer in view of resonance frequency, which itself is an indicator of implant stability.

### Self-Diagnostic Feature

In some embodiments, the sensor unit can include a self-diagnostic feature. Before each use of the clamp and MSU, the self-diagnostic feature can activate to determine if the sensor (e.g., the MEMS accelerometer) is functioning properly. In some examples, only after the sensor is confirmed to be properly functioning, can the forceps proceed to measure the angular stiffness. The self-diagnostic feature can help to reduce errors during use.

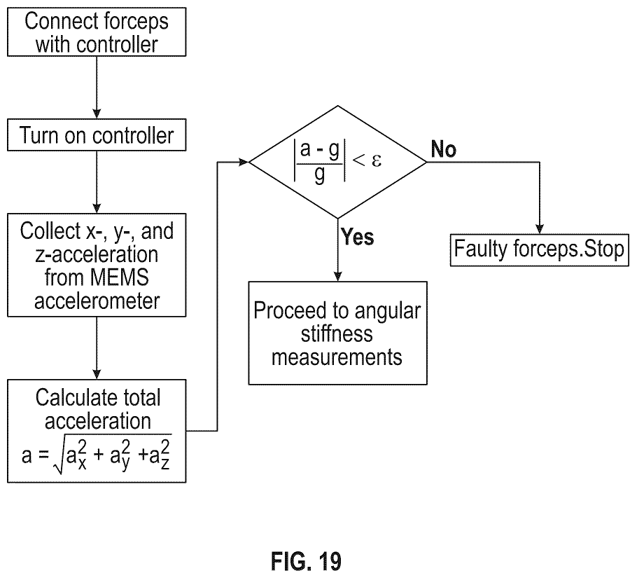

In some embodiments, the self-diagnostic feature is implemented by measuring the gravity using the MEMS accelerometer. FIG. 19 illustrates and embodiment of a flowchart of a self-diagnostic test. In some embodiments, a user first connects the forceps to the controller unit. Next, a controller can be turned on to power the sensor (e.g., accelerometer). In some embodiments, the controller can read the x-, y-, and z-acceleration components measured from the accelerometer, namely, aₓ, a_{y}, and a_{z}. In some embodiments, because the forceps can be placed in any orientation, any individual a_{y}, a_{y}, or a_{z} component does not measure the gravitational acceleration g. Instead, the resultant of all aₓ, a_{y}, and a_{z} can measure the gravitational acceleration g. Therefore, a resultant acceleration

$$a = \sqrt{{a}_{x}^{2} + {a}_{y}^{2} + {a}_{z}^{2}}$$

can be calculated and compared with the gravitational acceleration g, which is 9.80665 m/s². In some embodiments, if

$$\frac{\vert a - g \vert}{g} < ε ,$$

the sensor (e.g., MEMS accelerometer) is considered functional and accurate. The forceps can then proceed to measure angular stiffness. If not, the MEMS accelerometer is considered inaccurate, and the test stops.

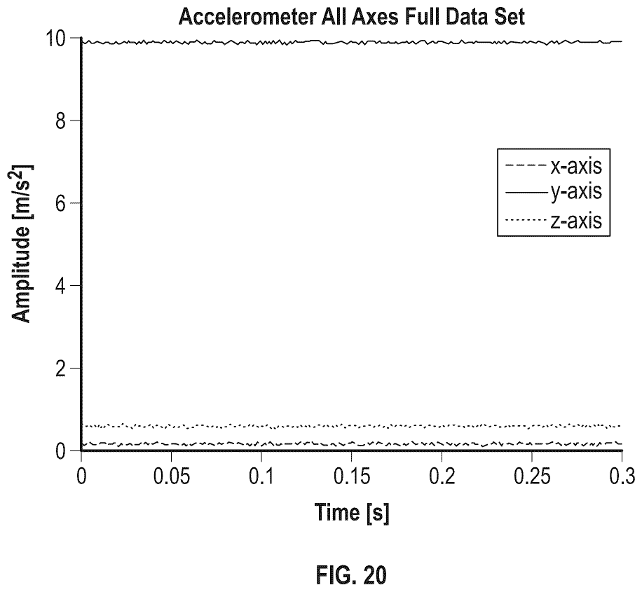

In some embodiments, ¿ is a pre-determined value, such as 3%. This can be a value chosen by users or manufacturers to accept an accelerometer. FIG. 20 shows an example of an accelerometer measurement. As shown, the measured a_{y}, a_{y}, or a_{z} are slightly fluctuating with respect to time. Table 1, provided below, shows the measurements at three sampled instants. In some embodiments, the difference between the measured acceleration and g is at most 1.1%. If a 3% error is acceptable, the forceps can proceed to measure angular stiffness.

TABLE 1
Three sample acceleration measurements

| aₓ | a_{y} | a_{z} | $a = \sqrt{{a}_{x}^{2} + {a}_{y}^{2} + {a}_{Z}^{2}}$ | $\frac{\vert a - g \vert}{g}$ |
|---|---|---|---|---|
| 0.60 | 9.85 | 0.11 | 9.87 | 0.61% |
| 0.58 | 9.88 | 0.16 | 9.90 | 0.93% |
| 0.56 | 9.90 | 0.15 | 9.92 | 1.1% |

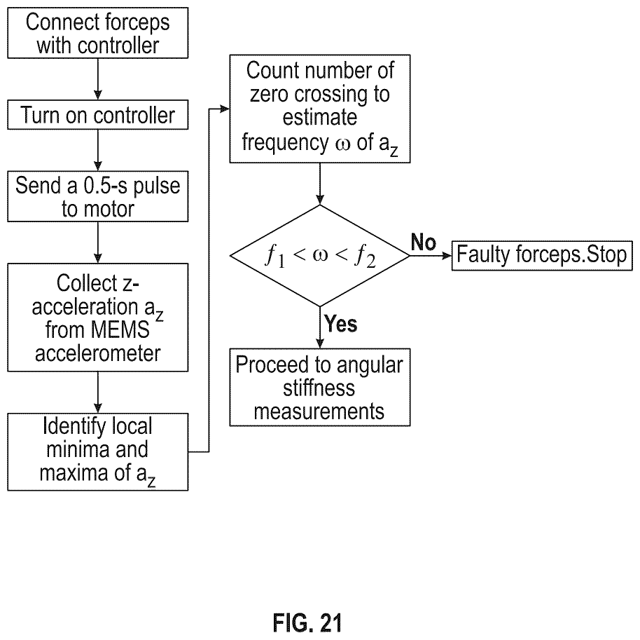

FIG. 21 illustrates an example of a flow chart for a self-diagnostic test. In some examples, a self-diagnostic feature can also be developed for the motor. This self-diagnostic feature can determine if the motor functions properly. In some embodiments, the self-diagnostic feature is simple and/or quantitative. For example, a simple the self-diagnostic feature can check if the motor spins. In some embodiments, an example of a quantitative self-diagnostic feature is detecting if the motor is spinning in an expected frequency range specified by a lower frequency f₁ and a higher frequency f₂.

In some examples, a user first connects the MSU to the controller unit (either via a hard wire or via a wireless link). The controller can then be turned on to power the motor and the accelerometer. In some examples, the controller sends to the motor a short pulse with a known voltage and a chosen duration (e.g., 0.5 seconds). In some embodiments, the acceleration a_{z} in the buccolingual direction is measured and recorded. In some examples, this can be from the z-component of the acceleration measured by the accelerometer.

In some embodiments, because the MEMS accelerometer is digital, the recorded measured a_{z} data can be a block of data with a fixed and known time increment, which is determined by the sampling rate of the accelerometer. In some examples, because the motor generates a sinusoidal force, the measured a_{z} data is therefore sinusoidal. Its frequency, however, is unknown but is expected to fall within a frequency range (f₁, f₂), such as 200 to 350 Hz, because the pulse voltage is known. In some examples, in order to extract the frequency, the measured a_{z} data can be analyzed at each time increment to identify a local maximum or a local minimum. Each transition from a local maximum to the next local minimum, and vice versa, constitutes a zero-crossing. Because the duration of the data block is known (e.g., 0.5 second), the number of zero-crossing can be translated to frequency using the following formula:

$$Frequency = ( no . of zero - crossings ) / ( 2 \times duration of the data block )$$

In some embodiments, other methods for extracting frequency can be used. For example, nonlinear regression analysis can be used to extract the frequency. In some examples, if the extracted frequency is within the expected frequency range (f₁, f₂), the motor is considered functional and acceptable. The self-diagnostic test is then completed, and the forceps can proceed to measure the angular stiffness. In some examples, if the extracted frequency is not within the expected frequency range, the forceps can be considered faulty and should be replaced.

### DISCLAIMERS

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount in certain embodiments that is within less than plus or minus 10% of, within less than plus or minus 5% of, within less than plus or minus 1% of, within less than plus or minus 0.1% of, and within less than plus or minus 0.01% of the stated amount or characteristic.

1. An intraoral sensor comprising:
a clamp, comprising:
an upper portion;
a lower portion comprising:
a plurality of holes for receiving forceps tips,
a first opening and a second opening, and
an engagement portion for contacting an implant;
a motor-sensor unit, comprising:
a force generator,
a sensor, and
a clip connecting the force generator and the sensor; and
wherein the intraoral sensor is configured to be retained on the implant to generate a measurement of implant stability;
wherein a first portion of the motor-sensor unit is received in the first opening of the lower portion of the clamp and a second portion of the motor-sensor unit is received in the second opening of the lower portion of the clamp; and
wherein the motor-sensor unit is removably connected to the clamp.

2. The intraoral sensor of claim 1, wherein the motor-sensor unit is secured to the clamp by an interference fit.

3. The intraoral sensor of claim 1 further comprising a pair of claws for engaging the implant.

4. The intraoral sensor of claim 3, wherein the motor-sensor unit further comprises at least one protrusion forming an axial stop, wherein the axial stop and the pair of claws are configured to secure the implant.

5. The intraoral sensor of claim 1, wherein the force generator comprises a motor.

6. The intraoral sensor of claim 1, wherein the sensor is an accelerometer.

7. The intraoral sensor of claim 1, wherein the implant is an abutment or a crown.

8. The intraoral sensor of claim 3, wherein the force generator is oriented to pass through a center of the pair of claws.

9. The intraoral sensor of claim 1, wherein the motor-sensor unit further comprises a clip extending between the force generator and the sensor, wherein the motor-sensor unit is elastic and allows the motor-sensor unit to open with the clamp.

10. The intraoral sensor of claim 1, wherein the clip of the motor-sensor unit is preloaded to help retain the motor-sensor unit on the clamp.

11. The intraoral sensor of claim 1, wherein the upper portion of the clamp is sufficiently tall to receive a pair of forceps while lowering a force required to open the pair of forceps.

12. The intraoral sensor of claim 3, wherein the claws of the clamp have an engagement portion that have a pair of straight edges joined at an obtuse angle, wherein the engagement portion is configured to accommodate an implant with a wide range of diameters.

13. The intraoral sensor of claim 1 further comprising an electrical cable extending from the sensor.

14. (canceled)

15. (canceled)

16. (canceled)

17. An intraoral sensor comprising:
a clamp, comprising:
an upper portion;
a lower portion comprising:
a plurality of holes for receiving forceps tips,
a first opening and a second opening, and
an engagement portion for contacting an implant;
a motor-sensor unit, comprising:
a motor,
an accelerometer,
a clip extending between the motor and the accelerometer, wherein the clip is preloaded and configured to retain the motor-sensor unit on the clamp, and
wherein the motor-sensor unit is secured to the clamp by an interference fit;
an electrical cable extending from the intraoral sensor; and
wherein the intraoral sensor is configured to be retained on the implant to generate a measurement of implant stability;
wherein a first portion of the motor-sensor unit is received in the first opening of the lower portion of the clamp and a second portion of the motor-sensor unit is received in the second opening of the lower portion of the clamp; and
wherein the motor-sensor unit is removably connected to the clamp.

18. An intraoral sensor comprising:
an engagement portion for contacting an implant;
a motor-sensor unit, comprising a force generator and a sensor; and
wherein the intraoral sensor is configured to be retained on the implant to generate a measurement of implant stability.
